(19) United States
(12) Patent Application Publication
(10) Pub. No.: US 2023/0360781 A1
(43) Pub. Date: Nov. 9, 2023

(54) METHOD AND SYSTEM FOR MANAGING MEDICAL CONSULTATION CONTENT

(71) Applicant: IKOOB CO.,LTD, Seoul (KR)

(72) Inventors: Yeop HYUN, Hwaseong-si (KR); Young In KIM, Seoul (KR)

(21) Appl. No.: 18/013,664

(22) PCT Filed: Jun. 17, 2021
(86) PCT No.: PCT/KR2021/007583

§ 371 Date: Dec. 29, 2022

(30) Foreign Application Priority Data
Jun. 29, 2020 (KR) 10-2020-0079413

(51) Int. Cl. G16H 40/20; G16H 10/60; G16H 10/20; G16H 80/00

(52) U.S. Cl. CPC G16H 40/20; G16H 10/60; G16H 10/20; G16H 80/00

(57) ABSTRACT

A method and a system for managing medical consultation content, according to one embodiment of the present disclosure, relate to a method by which a processor of a counselee terminal manages medical consultation content of a counselee, comprising the steps of: acquiring the medical consultation content of the counselee through at least one medium of an offline printout and/or an online printout; acquiring counselee information matched with the medical consultation content if the counselee information matched with the acquired medical consultation content does not exist; matching the acquired counselee information with the medical consultation content and storing the same; controlling a display to output the medical consultation content matched with the counselee information; and managing the stored medical consultation content on the basis of a medical consultation application.

### BACKGROUND

### Field

The present disclosure relates to a method and a system for managing medical consultation content. More particularly, the present disclosure relates to a method and a system for systematically managing medical consultation content for a plurality of counselees by matching the same for each counselee.

### Related Art

In general, the exchange of information of medical treatment about patients is necessary in various situations such as when a patient is transferred from a lower medical institution (for example, primary or secondary medical institutions such as private hospitals or small and medium-sized hospitals) to an upper medical institution (for example, university hospitals, general hospitals, etc.), or when a patient medical request (transfer) is performed between medical institutions in different medical fields.

Conventionally, the exchange of medical treatment information is performed in the form of a patient taking a memo of medical treatment information received from a previous medical institution or accessing a homepage of the previous medical institution hospital to request or inquire about related data.

However, the exchange of medical treatment information in this way is vulnerable to a security breach and is merely a concept of information inquiry, not information exchange, so detailed delivery of information is impossible, and there is a difficulty in that a patient has to perform a series of procedures to acquire his or her own medical treatment information.

Meanwhile, with the development of information technology (IT), various technologies and systems for providing customized medical services specialized to each patient are being developed and introduced.

For example, these digital systems record the condition or medical treatment process of a patient as objective data such as an Electronic Medical Record (EMR) or Electronic Health Record (EHR), and enable utilization and data processing in a wide range of fields based on the recorded data.

However, according to the current medical law, it is difficult to share data with the private sector because actions such as storing and extracting medical treatment information such as the EMR must be performed only within medical institutions, which makes it difficult for patients to access even though it is their own medical treatment information.

In addition, medical institutions where patients are treated perform diagnosis based on the EMR formed in a standardized manner, but demand for base data that may assist patients' treatment in a more diversified way other than the EMR is emerging.

In addition, in the past, technology capable of autonomously managing medical treatment information of oneself is insufficient from the viewpoint of a patient rather than the viewpoint of a medical institution, and a solution thereto is required.

In addition, a situation may arise in which a guardian of a patient has to manage the medical treatment information of the subject patient, such as when the patient is a minor, but the technology therefor is also insufficient, resulting in inconvenience.

### SUMMARY

An aspect of the present disclosure is to provide a method and a system for managing medical consultation content capable of safely and easily sharing and managing medical treatment information of a patient.

In detail, an aspect of the present disclosure is designed to provide a method and a system for managing medical consultation content in which a patient is able to autonomously acquire and manage his or her medical treatment information generated offline on a terminal.

In addition, an aspect of the present disclosure is designed to provide a method and a system for managing medical consultation content in which a guardian of an authenticated patient is able to acquire and manage medical treatment information of a patient.

In addition, an aspect of the present disclosure is designed to provide a method and a system for managing medical consultation content capable of generating and providing medical treatment information of a patient capable of replacing an Electronic Medical Record (EMR), which is medical treatment information that is difficult to share with the private sector under the current medical law.

Technical aspects to be achieved by the present disclosure and embodiments according to the present disclosure are not limited to the technical aspects described above, and other technical aspects may also be addressed.

A method and a system for managing medical consultation content, according to an embodiment of the present disclosure, relate to a method by which a processor of a counselee terminal manages medical consultation content of a counselee, wherein the method includes: acquiring the medical consultation content of the counselee through at least one medium of an offline printout and/or an online printout; acquiring counselee information matched with the medical consultation content when the counselee information matched with the acquired medical consultation content does not exist; matching the acquired counselee information with the medical consultation content and storing the same; controlling a display to output the medical consultation content matched with the counselee information; and managing the stored medical consultation content on the basis of a medical consultation application.

In this connection, the counselee information is information for deciding a counselee identification and an owner of the medical consultation content, and includes at least one of a counselee name, gender, age, resident registration number, service account information, and terminal identification number.

In addition, the acquiring of the counselee information matched with the medical consultation content includes: acquiring the counselee information through a counselee information input interface provided through a web browser; or acquiring the counselee information pre-stored through member registration of the medical consultation application.

In addition, the acquiring of the medical consultation content of the counselee through the offline printout includes: recognizing an identification code included in the offline printout; and acquiring the medical consultation content matched with the recognized identification code, wherein the identification code, as a medium for identifying the medical consultation content, is any one of a QR code, a barcode, or a unique identification number.

In addition, the controlling of the display to output the medical consultation content matched with the counselee information further includes: when the medical consultation application is presently installed in the counselee terminal, outputting the medical consultation content by operating the medical consultation application; or when the medical consultation application is not installed in the counselee terminal, outputting the medical consultation content in a form of a text or providing an installation process for the medical consultation application.

The method further includes providing a member registration interface for the medical consultation application and acquiring the counselee information when installation of the medical consultation application is completed based on the installation process for the medical consultation application.

In addition, the managing of the stored medical consultation content on the basis of the medical consultation application includes providing a medical consultation content management function including classification management, deletion management, and counselee information interlock management functions for the at least one piece of medical consultation content.

In addition, the managing of the stored medical consultation content on the basis of the medical consultation application further includes providing a member registration interface, wherein the member registration interface is an interface for registering the counselee information for a plurality of counselees by adding at least one counselee other than the counselee to the medical consultation application as members.

In addition, the managing of the acquired medical consultation content on the basis of the medical consultation application further includes providing an owner selection interface, wherein, upon receiving the medical consultation content on the basis of the medical consultation application, the owner selection interface is an interface that matches the received medical consultation content with the counselee information of any one of the plurality of counselees registered in the medical consultation application on the basis of the member registration interface.

In addition, the counselee information interlock management further includes a function of selecting the counselee information matched with each of the medical consultation content existing in the medical consultation application on the basis of the counselee information for the plurality of counselees when the plurality of counselees are registered in the medical consultation application.

The method and system for managing medical consultation content according to an embodiment of the present disclosure enable extension of the available range of patient medical treatment information by safely and easily sharing and managing medical treatment information of patients.

In addition, the method and system for managing medical consultation content according to an embodiment of the present disclosure enable patients to acquire and manage their own medical treatment information, so that not only hospitals but also patients themselves are able to check and manage their own medical treatment information. Also, when a patient is transferred to another hospital, the convenience of exchanging medical treatment information for patients can be increased.

In addition, the method and system for managing medical consultation content according to an embodiment of the present disclosure enable a guardian of an authenticated patient to acquire and manage the medical treatment information of the patient, so that when the guardian needs to manage the patient, such as when the patient is a minor, the guardian of the patient is able to check and manage the medical treatment information of the patient, and thus, more effective patient care can be assisted.

In addition, the method and system for managing medical consultation content according to an embodiment of the present disclosure generate and provide medical treatment information of a patient that can replace the EMR, which is medical treatment information that is difficult to share with the private sector under the current medical law. Accordingly, accessibility to the medical treatment information of the patient can be improved, and utilization of patient medical treatment information can be enhanced.

In addition, the method and system for managing medical consultation content according to an embodiment of the present disclosure generate and provide medical treatment information of a patient that can replace the EMR, thus enabling use of base data that may assist patients' treatment in a more diversified way other than the EMR formed in a standardized manner in medical institutions where patients are treated.

The benefits of the present disclosure are not limited to those mentioned above, and other benefits not mentioned herein will be clearly understood by those skilled in the art from the following description.

### BRIEF DESCRIPTION OF THE DRAWINGS

FIG. 1 is a conceptual diagram of a system for managing medical consultation content according to an embodiment of the present disclosure.

FIG. 2 is an internal block diagram of a terminal according to an embodiment of the present disclosure.

FIG. 3 shows an example of an external appearance of a terminal according to an embodiment of the present disclosure.

FIG. 4 is an internal block diagram of a medical consultation content management server according to an embodiment of the present disclosure.

FIG. 5 is a flowchart illustrating a method for managing medical consultation content in a counselor terminal according to an embodiment of the present disclosure.

FIG. 6 is an example of an offline printout according to an embodiment of the present disclosure.

FIG. 7 is a flowchart illustrating a method for managing medical consultation content when a single counselee exists in a counselee terminal according to an embodiment of the present disclosure.

FIG. 8 is a diagram for explaining a method for acquiring medical consultation content on the basis of an identification code of an offline printout according to an embodiment of the present disclosure.

FIG. 9 is an example showing a process of outputting medical consultation content as an online printout according to an embodiment of the present disclosure.

FIG. 10 is a flowchart illustrating a method for managing medical consultation content when a plurality of counselees exist in a counselee terminal according to an embodiment of the present disclosure.

FIG. 11 is an example of a member registration interface according to an embodiment of the present disclosure.

FIG. 12 is an example of an owner selection interface according to an embodiment of the present disclosure.

### DESCRIPTION OF EXEMPLARY EMBODIMENTS

Embodiments of the present disclosure will be shown in the drawings and described in detail through a detailed description. However, the present disclosure may be variously modified and embodied in a variety of different embodiments. Advantages and features of the present disclosure and implementation methods thereof will be clarified through the following embodiments described with reference to the accompanying drawings. Therefore, the present disclosure is not limited to these embodiments introduced hereinafter and might be embodied in a different shape from these embodiments. The terms “first”, “second”, and so on in the present disclosure are used for distinguishing one component from the other components, but they do not specify limited meanings. Also, the singular forms used in the present disclosure are intended to include the plural forms, unless the context clearly indicates otherwise. Moreover, the terms “comprises” and/or “having” described in the present disclosure specify the presence of stated components and/or features, but do not preclude the presence or addition of one or more other components and/or features. Furthermore, the size or the thickness of each component in the drawings can be exaggerated or reduced for the definiteness of explanation. For example, the size and the thickness of each component in the drawings are arbitrarily represented for the convenience of explanation. In accordance therewith, the present disclosure is not limited to the matters shown in the drawings.

Reference will now be made in detail to the embodiments of the present disclosure with reference to the accompanying drawings. Wherever possible, the same reference numbers will be used throughout the disclosure including the drawings to refer to the same or like parts. As such, the repeatable description of the same or like parts will be omitted.

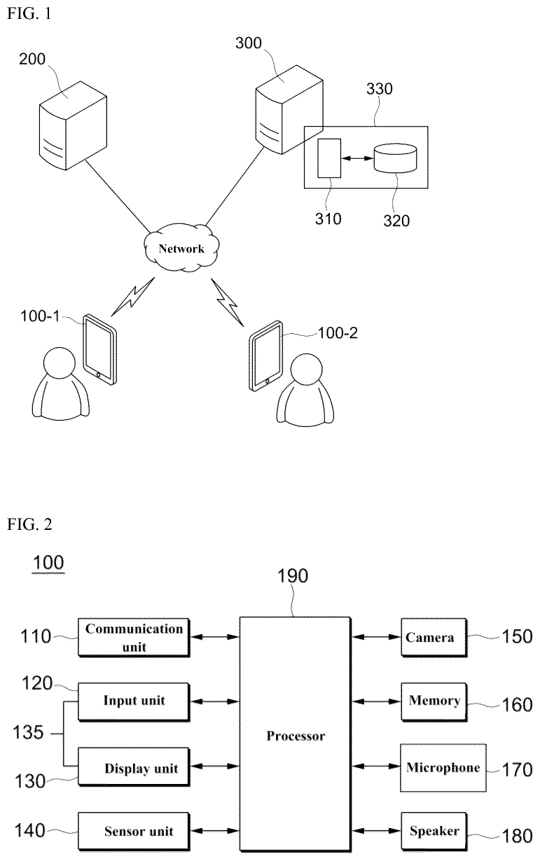

FIG. 1 is a conceptual diagram of a system for managing medical consultation content according to an embodiment of the present disclosure.

Referring to FIG. 1, the system for managing medical consultation content according to an embodiment of the present disclosure may include a terminal 100, a medical consultation content management server 200, and an electronic medical record management server 300.

In an embodiment of the present disclosure, the terminal 100, the medical consultation content management server 200, and the electronic medical record management server 300 may acquire medical consultation content for a plurality of counselees by interoperating with one another, and provide a service that systematically manages the acquired medical consultation content by matching the same to each counselee.

Here, each component of FIG. 1 may be connected through a network. The network refers to a connection structure capable of exchanging information between nodes such as the terminal 100, the medical consultation content management server 200, and the electronic medical record management server 300. Examples of the network include 3GPP (3rd Generation Partnership Project) network, LTE (Long Term Evolution) network, WIMAX (World Interoperability for Microwave Access) network, Internet, LAN (Local Area Network), Wireless LAN (Wireless Local Area Network), WAN (Wide Area Network), PAN (Personal Area Network), Bluetooth network, Satellite Broadcasting Network, Analog Broadcasting Network, and DMB (Digital Multimedia Broadcasting) network, but are not limited thereto.

Terminal

First, in an embodiment of the present disclosure, the terminal 100 may include a smart phone, a digital broadcasting terminal 100, a mobile phone, a PDA (personal digital assistant), a PMP (portable multimedia player), a navigation device, a tablet PC, a wearable device, and smart glasses, which are portable terminals in which a program for performing a medical consultation content management service is installed.

In addition, the terminal 100 may further include devices in which a program for executing the medical consultation content management service based on wired/wireless communication, such as personal computers is installed, which include a desktop PC, which is a fixed terminal, a laptop computer, and an ultrabook.

The role of the terminal 100 may be divided according to the subject used in an embodiment of the present disclosure.

In detail, in an embodiment, the terminal 100 may be divided into a counselor terminal 100-1 used by a counselor (doctor) and a counselee terminal 100-2 used by a counselee (patient).

First, the counselor terminal 100-1 may acquire medical treatment information about a counselee, and may generate and provide medical consultation assistant content based on the acquired medical treatment information.

Herein, the medical treatment information may be information related to a medical treatment of a patient formed by a combination including at least one piece of information of Electronic Medical Records (EMRs), medical-related images, videos, animations, and texts for a counselee.

In addition, the medical consultation assistant content is information that may assist medical consultation by being formed in a combination including at least one of medical images, videos, animations, and texts and output through the terminal 100, and may include EHR, EMR or images assisting in consultation provided by the medical consultation content management server 200.

In detail, the medical consultation assistant content provided by the medical consultation content management server 200 may include audio-visual data for assisting medical consultation. In an embodiment, the audio-visual data may include disease diagnosis content, treatment method content, medicine consultation content, medical treatment cost content, insurance information content, sign content, other content, and medical consultation assistant content combining the aforementioned content.

For example, the medical consultation assistant content may include at least one image or at least one piece of information of body organ images, disease information, treatment method images, treatment method information, medicine-taking method, medicine information, and insurance information, and may additionally include various pieces of information that are used to construct the medical consultation content.

In addition, in an embodiment, the medical consultation assistant content may be visual content generated on the basis of medical treatment information of a counselee.

Such various pieces of the medical consultation assistant content are displayed on the terminal 100 to be used for medical consultation in accordance with selection of a counselor (a doctor), and the terminal 100 may arrange and provide a plurality of pieces of selected medical consultation assistant content for a counselor to easily utilize the same.

In addition, the counselor terminal 100-1 may provide a consultation input interface based on the generated medical consultation assistant content.

Herein, the consultation input interface may include a graphic user interface that enables a counselor input writing, voice, editing, and/or images to the displayed image of the medical consultation assistant content.

For example, the consultation input interface may provide a writing input interface that detects writing input of a doctor on the image of the medial consultation assistant content.

In addition, the counselor terminal 100-1 may generate medical consultation content, which is information generated by adding a consultation detail record input through the consultation input interface to the medical consultation assistant content.

In other words, the medical consultation content may be information generated by adding the medical consultation content input through the consultation input interface to the aforementioned consultation assistant content, and is output through the terminal 100 to play a role in assisting medical consultation.

In this connection, the counselor terminal 100-1 may generate, match, and manage an identification code that matches the medical consultation content.

Herein, the identification code is a medium for identifying each piece of the medical consultation content, and may be, for example, a QR code, a barcode, or a unique identification number.

In addition, the counselor terminal 100-1 may provide the generated medical consultation content as an offline printout and/or an online printout.

In an embodiment of the present disclosure, the counselee terminal 100-2 may acquire the medical consultation content, and may output and provide the acquired medical consultation content.

In addition, the counselee terminal 100-2 may drive a medical consultation content management application (hereinafter referred to as a medical consultation application) that provides a medical consultation content management service, thereby providing a medical consultation content management function through the medical consultation application.

The roles of the counselor terminal 100-1 and the counselee terminal 100-2 as described above are divided according to the subject of use, and functional characteristics performed by the terminal 100 may be the same.

Hereinafter, each component configuring the terminal 100 will be described in detail with reference to the accompanying drawings.

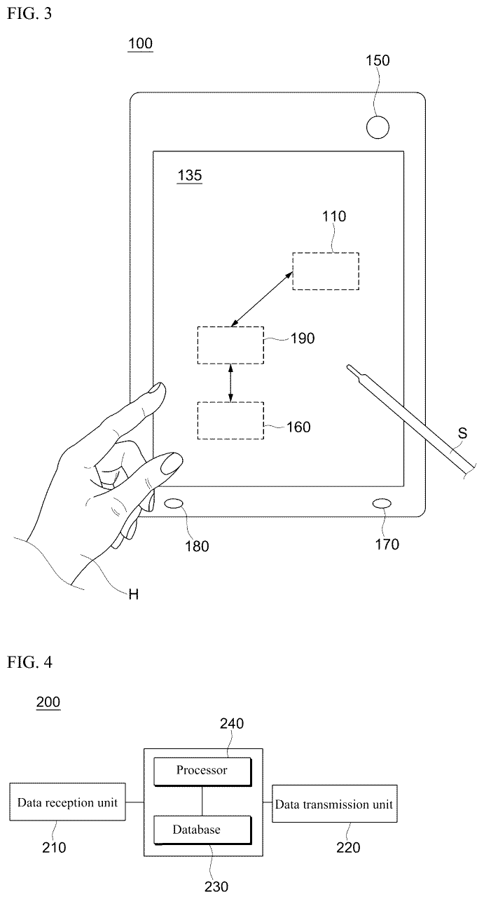

FIG. 2 is an internal block diagram of the terminal 100 according to an embodiment of the present disclosure. FIG. 3 shows an example of an external appearance of the terminal 100 according to an embodiment of the present disclosure.

Referring to FIGS. 2 and 3, the terminal 100 may include a communication unit 110, an input unit 120, a display unit 130, a touch screen 135, a sensor unit 140, a camera 150, a memory 160, a microphone 170, a speaker 180, and a processor 190.

First, the communication unit 110 may transmit and receive various types of data and/or information to provide the medical consultation content management service.

In an embodiment, the communication unit 110 may communicate with an external terminal 100 and/or the medical consultation content management server 200 to transmit/receive data related to the medical consultation content management service.

In addition, the communication unit 110 may transmit the medical consultation content to an external output device (for example, a printer) and assist in printing the same as offline printout.

The communication unit 120 may transmit/receive wireless signals to/from at least one of a base station, the external terminal 100, and a certain server on a mobile communication network constructed in accordance with technical standards or communication methods for mobile communication (for example, GSM (Global System for Mobile communication), CDMA (Code Division Multi Access), HSDPA (High Speed Downlink Packet Access), HSUPA (High Speed Uplink Packet Access), LTE (Long Term Evolution), and LTE-A (Long Term Evolution-Advanced)).

Next, the input unit 120 may detect a user input related to the medical consultation content management service.

As an embodiment, the input unit 120 may detect the user input through the consultation input interface, receive a signature of a counselee (patient) and/or a counselor (doctor), and detect an input to a destination for sending the medical consultation content.

Next, the display unit 130 may output various items of information related to the medical consultation content management service as a graphic image.

The display unit 130 may include at least one of a liquid crystal display (LCD), a thin film transistor liquid crystal display (TFT LCD), an organic light emitting diode (OLED), a flexible display, a 3D display, and an e-ink display.

In addition, the input unit 120 and the display unit 130 may be combined to be implemented as the touch screen 135.

Next, the camera 150 may capture medical treatment and consultation processes to acquire an image of the medical consultation content, and the terminal 100 that has acquired the image may include the acquired image in the medical consultation assistant content and/or medical consultation content.

In addition, the camera 150 may be disposed on the front or/or rear side of the terminal 100 to acquire an image by capturing the disposed direction side, and may be disposed outside the terminal 100 to capture the medical treatment process from an external viewpoint.

When the camera 150 is disposed outside the terminal 100, the camera 150 may send an image of the captured medical treatment process to a processor 190 through the communication unit 110.

In addition, the camera 150 may include an image sensor and an image processing module.

In detail, the camera 170 may process still images or videos which are acquired by an image sensor (for example, CMOS or CCD).

In addition, the camera 150 may process a still image or video acquired through an image sensor using an image processing module to extract necessary information and transfer the extracted information to the processor 190.

Next, the memory 160 may store any one or more of various application programs, data, and commands for providing the medical consultation content management service according to an embodiment of the present disclosure.

In an embodiment, the memory 160 may store and manage various types of medical treatment information, medical consultation assistant content, and/or medical consultation content.

The memory 160 may be various storage devices such as an ROM, an RAM, EPROM, a flash drive, and a hard drive, and may be a web storage that performs the storage function of the memory 160 on the Internet.

Next, the sensor unit 140 may sense fingerprint information of a patient and/or a counselor, and may assist in proceeding a signature consent procedure based on the sensed fingerprint information.

Next, the microphone 170 may detect a voice input of a patient and/or a counselor during a medical consultation process, and may acquire consultation content recording information based on the detected voice.

In this connection, the terminal 100 that has acquired the consultation content recording information may include the acquired recording information in the medical consultation content.

Next, the speaker 180 may output audio information related to the medical consultation content management service.

In an embodiment, the speaker 180 may output and provide the consultation content recording information included in medical consultation content.

Finally, the processor 190 may control the overall operation of each of the components described above in order to provide the medical consultation content management service.

The processor 190 may be implemented using at least one of application specific integrated circuits (ASICs), digital signal processors (DSPs), digital signal processing devices (DSPDs), programmable logic devices (PLDs), field programmable gate arrays (FPGAs), controllers, micro-controllers, microprocessors, and electronic units for performing other functions.

However, the components illustrated in FIGS. 2 and 3 are not essential to implement the terminal 100, so the terminal 100 described in this specification includes more or fewer components than the components listed above.

Medical Consultation Content Management Server

Next, in an embodiment of the present disclosure, the medical consultation content management server 200 (hereinafter referred to as the medical consultation server) may match the medical consultation content of a counselee with counselee information for identifying the corresponding counselee, form a database and store the same, and provide the corresponding information upon request from the terminal 100.

In addition, the medical consultation server 200 may provide an assistant content search function through which a counselor may search for and select necessary medical consultation assistant content during medical consultation, and may transmit and provide medical consultation assistant content selected by the counselor to the counselor terminal 100-1.

FIG. 4 is an internal block diagram of the medical consultation content management server 200 according to an embodiment of the present disclosure.

Referring to FIG. 4, the aforementioned medical consultation server 200 according to an embodiment of the present disclosure may include a data reception unit 210, a data transmission unit 220, a database 230, and a data processing unit 240.

First, the data reception unit 210 and the data transmission unit 220 may receive various types of data for performing the medical consultation content management service, such as the terminal 100 and/or an electronic medical record server, through a network.

In an embodiment, the data reception unit 210 may acquire medical consultation content and counselee information matched with the corresponding medical consultation content from the terminal 100 through a network, and the data transmission unit 220 may send the corresponding information read from a database 230 to the corresponding terminal 100 through a network upon request of the terminal 100.

Next, the database 230 may store and manage various types of data related to the medical consultation content management service.

In an embodiment, the database 230 may store and manage the medical consultation assistant content, medical consultation content, counselee information, and/or counselee account information.

The database 140 may be various storage devices such as a ROM, a RAM, an EPROM, a flash drive, and a hard drive, and may be a web storage that performs the storage function of the database 140 on the Internet.

Finally, the data processing unit 240 may control the overall operation of each of the components described above in order to implement the medical consultation content management service.

The data processing unit 240 may be implemented using at least one of the ASICs, DSPs, DSPDs, PLDs, FPGAs, controllers, micro-controllers, microprocessors, and electronic units for performing other functions.

In addition, in an embodiment of the present disclosure, it is described that the medical consultation server 200 performs the aforementioned functions such as matching the medical consultation content of a counselee with the counselee information capable of identifying the corresponding counselee, forming the database 230, and providing a medical consultation assistant content search function. However, in other embodiments, various embodiments may be possible, such as performing some operations of the functions performed by the medical consultation server 200 in the terminal 100.

Electronic Medical Record Management Server

In an embodiment of the present disclosure, the electronic medical record management server 300 may store and manage the electronic medical record of a counselee by matching it with individual information of the corresponding counselee.

Herein, the Electronic Medical Record (EMR) is a computerized patient chart written on existing paper, and may be data information including at least one of personal data of a patient, past medical history, diagnosis record, treatment details, medication guidance record, medication intake record, surgery record, hospitalization and discharge records, and outpatient medical treatment situation information.

The electronic medical record may be stored in the electronic medical record management server 300 for each hospital because security of information is important for individual privacy protection.

In detail, the electronic medical record management server 300 may convert at least a portion of the individual information and the electronic medical record of a counselee into hashes and store the same in order to protect the privacy of the counselee. In this connection, the electronic medical record may be converted and stored using an account of the counselee as a key, and the hash may be stored in a searchable manner based on the account of the counselee.

Thereafter, when the electronic medical record management server 300 may search for and send the hashed electronic medical record by matching the same with the hash when the medical consultation server 200 and/or the counselor terminal 100-1 request(s) the electronic medical record through an account of a counselee.

The hashed electronic medical record may be decrypted with account information of a counselee as a key, and the decrypted electronic medical record may be output on the counselor terminal 100-1 in the form of the captured medical consultation assistant content.

Referring further to FIG. 1, in an embodiment of the present disclosure, the aforementioned electronic medical record server may include a data transmission/reception unit 310, a storage unit 320, and a control unit 330.

First, the data transmission/reception unit 310 may transmit/receive data for providing the medical consultation content management service with the terminal 100 and/or an external server through a network.

In addition, the storage unit 320 may store electronic medical records for each counselee in a searchable manner. In an embodiment, the storage unit 320 may store an account of the counselee and electronic medical records matched with the corresponding account.

The storage unit 320 may be various storage devices such as an ROM, an RAM, EPROM, a flash drive, and a hard drive.

Finally, the control unit 330 may process data to provide the medical consultation content management service. The control unit 330 may be implemented using at least one of the ASICs, DSPs, DSPDs, PLDs, FPGAs, controllers, micro-controllers, microprocessors, and electronic units for performing other functions.

Method for Managing Medical Consultation Content in Counselor Terminal

Hereinafter, a method for managing medical consultation content in the counselor terminal 100-1 according to an embodiment of the present disclosure will be described in detail with reference to the accompanying drawings.

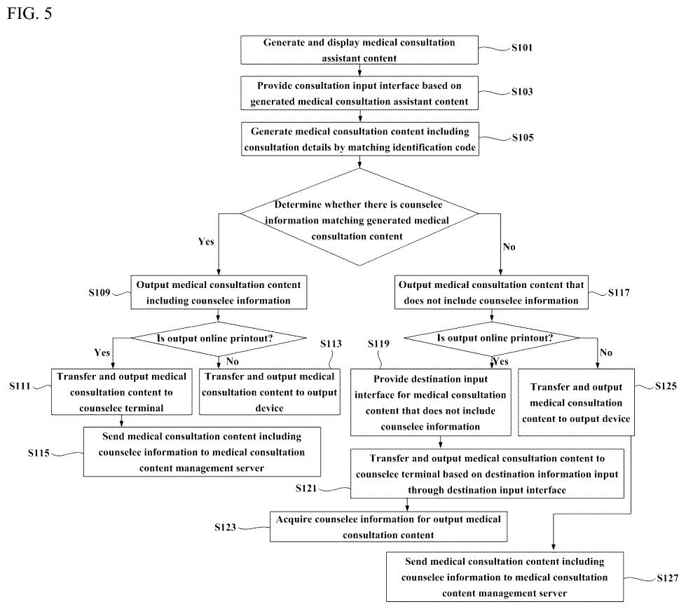

FIG. 5 is a flowchart illustrating a method for managing medical consultation content in the counselor terminal 100-1 according to an embodiment of the present disclosure.

Referring to FIG. 5, in an embodiment of the present disclosure, the counselor terminal 100-1 may first acquire medical treatment information about a counselee, generate medical consultation assistant content, and display the generated medical consultation assistant content (S101).

Herein, the medical treatment information according to an embodiment may be information related to a medical treatment of a patient formed by a combination including at least one piece of information of electronic medical records of a counselee, medical-related images, videos, animations, and texts.

Further, in an embodiment, the medical consultation assistant content may be information that may assist medical consultation by being generated based on the medical treatment information of a counselee and output through the terminal 100.

The counselor terminal 100-1 that has generated such medical consultation assistant content may provide a consultation input interface based on the generated medical consultation assistant content (S103).

Herein, the consultation input interface may include a graphic user interface that enables a counselor input writing, voice, editing, and/or images on the medical consultation assistant content.

For example, the consultation input interface may provide a writing input interface that detects writing input of a doctor on the image of the medial consultation assistant content.

Next, the counselor terminal 100-1 may generate the medical consultation content including consultation details input on the medical consultation assistant content through the consultation input interface by matching an identification code (S105).

Herein, the identification code is a medium for identifying each piece of the medical consultation content, and may be, for example, a QR code, a barcode, or a unique code for the medium, such as a plurality of listed numbers.

In other words, the counselor terminal 100-1 may generate medical consultation content to which a record of consultation details input through the consultation input interface is added to the medical consultation assistant content, generate and store an identification code matching the generated medical consultation content, and thus, manage each piece of the medical consultation content in correspondence with the identification code.

In this connection, in order to ensure that the medical consultation content generated by a plurality of counselor terminals have a unique identification code, the medical consultation server 200 may assign a unique identification code to the medical consultation content when the medical consultation content is generated in the counselor terminal 100-1.

Next, the counselor terminal 100-1 may determine whether there is counselee information matching the generated medical consultation content (S107).

Herein, the counselee information is information for determining the counselee identification and the owner of the medical consultation content, and includes at least one of a counselee account, name, gender, age, resident registration number, and/or terminal identification number (phone number).

Such counselee information may be interlocked with service account information generated by matching each counselee when signing up for a medical consultation application that provides the medical consultation content management service.

In an embodiment, the service account information may include counselee information and/or counselee account ID.

In detail, the counselor terminal 100-1 may check whether counselee information corresponding to the medical consultation content including an identification code specifying each piece of medical consultation content exists.

In this connection, when counselee information or/and service account information corresponding to the medical consultation content exists, the counselor terminal 100-1 may match the corresponding medical consultation content with the corresponding counselee information and store the same in the memory 160, and may output the medical consultation content including the counselee information (S109)

In detail, the counselor terminal 100-1 may output the medical consultation content including the counselee information as an online printout or an offline printout.

Herein, the online printout according to an embodiment may refer to a printout displayed as a graphic image based on digital machinery or device.

In addition, the offline printout may refer to a printout printed on paper or cloth through an output device (for example, a printer) and displayed thereon.

In more detail, when the counselor terminal 100-1 first provides the medical consultation content as an online printout, the medical consultation content including the counselee information may be transmitted to the counselee terminal 100-2 of the corresponding counselee based on the counselee information and/or account information interlocked to the counselee information (S111).

Herein, the counselor terminal 100-1 may provide the counselee terminal 100-2 with medical consultation content including counselee information either through texting or a medical consultation application method.

For example, the counselee terminal 100-1 may transmit medical consultation content to the counselee terminal 100-2 using texting based on the terminal identification number of a counselee.

In this connection, when the medical consultation content is sent using texting, the counselor terminal 100-1 may generate a link to provide the corresponding medical consultation content as a graphic image, and may include the generated link in the corresponding text message and transmit the same.

Thus, the counselor terminal 100-1 may output the medical consultation content matched with the corresponding link as a graphic image when a selection input for the link of the text message received from the counselee terminal 100-2 is detected.

In another embodiment, the counselor terminal 100-1 may send medical consultation content through a medical consultation application installed in the corresponding counselee terminal 100-2 based on an account ID of a counselee.

Herein, the counselor terminal 100-1 transmits medical consultation content through a medical consultation application installed in the corresponding counselee terminal 100-2 when the medical consultation application is presently installed in the corresponding counselee terminal 100-2. Accordingly, transmission of medical consultation content may be notified through a pop-up message of the medical consultation application.

When the medical consultation application is not installed in the corresponding counselee terminal 100-2, the counselor terminal 100-1 may provide a link including a medical consultation application installation process for installing the medical consultation application on the corresponding counselee terminal 100-2 through text/SNS message or the like.

For example, the counselor terminal 100-1 may first determine whether a medical consultation application is installed in the counselee terminal 100-2. In an embodiment, when the counselor terminal 100-1 is matched with the account information of a counselee and transmits the medical consultation content to the medical consultation server 200, the medical consultation server 200 sends medical consultation content to the counselee terminal 100-2 matched with the account information of the counselee. In this connection, the counselee terminal 100-2 may transmit the medical consultation content in different ways depending on whether the medical consultation application is installed.

In detail, when it is determined that the medical consultation application is not installed, the medical consultation server 200 may forward a text message including a link to a medical consultation application installation page to the corresponding counselee terminal 100-2 based on the terminal identification number of the corresponding counselee. Thereafter, when a selection input for the link of the text message received from the counselee terminal 100-2 is detected, an installation process may be provided by moving to the medical consultation application installation page.

In addition, the medical consultation server 200 may output the corresponding medical consultation content through the installed medical consultation application when installation of the medical consultation application is completed in the corresponding counselee terminal 100-2. A detailed description thereof will be described below.

As described above, the counselor terminal 100-1 may control the display unit 130 to output the medical consultation content received from the counselee terminal 100-2 that has received the medical consultation content.

When the medical consultation content is provided as offline printout, the counselor terminal 100-1 may transfer and output the medical consultation content including counselee information to an external output device (for example, a printer) (S113)

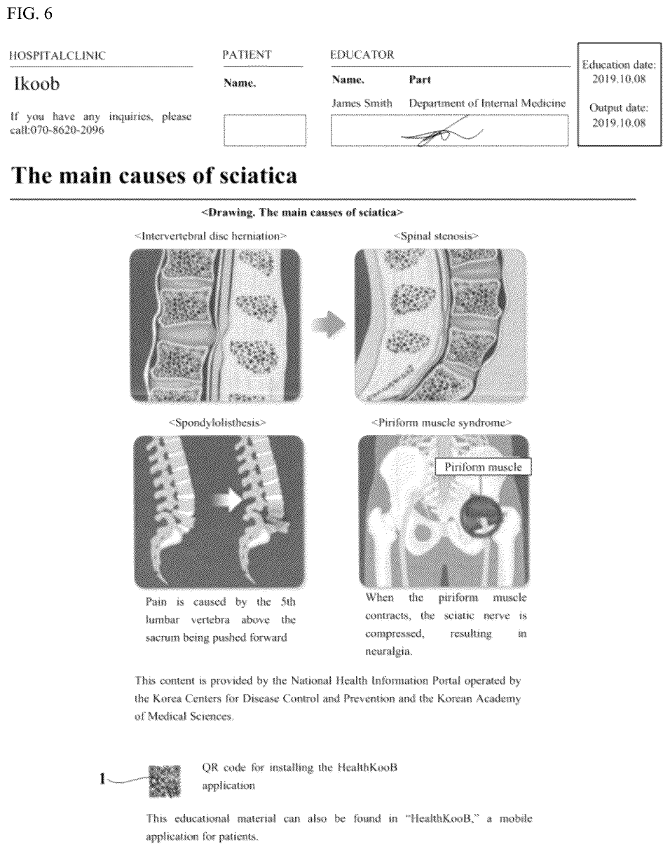

FIG. 6 is an example of an offline printout according to an embodiment of the present disclosure.

Referring to FIG. 6, in this connection, the counselor terminal 100-1 may display and output an offline printout of medical consultation content output through an external output device together with a predetermined identification code 1 (for example, a QR code) matching the corresponding medical consultation content.

Herein, when the terminal 100 detects the identification code 1 displayed on the offline printout through the camera 150, the identification code 1 may execute a medical consultation application installed in the corresponding terminal 100 to output medical consultation content matching the corresponding identification code 1. In addition, the identification code 1 may provide a medical consultation application installation process when the medical consultation application is not installed in the corresponding terminal 100. A detailed description thereof will be described later.

In addition, the counselor terminal 100-1 may send and provide medical consultation content including counselee information to the medical consultation server 200 (S115).

In other words, the counselor terminal 100-1 sends the medical consultation content matched with the counselee information to the medical consultation server 200, so that the medical consultation server 200 may construct the database 230 capable of managing medical consultation content for each counselee.

The counselor terminal 100-1 may provide medical consultation content that does not include counselee information when counselee information corresponding to the medical consultation content does not exist (S117).

In detail, the counselor terminal 100-1 may output medical consultation content that does not include counselee information as an online printout or an offline printout.

First, in the case of outputting medical consultation content that does not include counselee information as an online printout, the counselor terminal 100-1 may provide a separately provided destination input interface to transfer the medical consultation content that does not include the counselee information to the external terminal 100 (S119).

In an embodiment, the counselor terminal 100-1 may provide a destination input interface through which a terminal identification number and/or a counselee account ID may be input.

Further, the counselor terminal 100-1 may transmit medical consultation content that does not include counselee information to the specific counselee terminal 100-2 based on the destination information input through the destination input interface (S121).

In an embodiment, the counselor terminal 100-1 may acquire the terminal identification number and/or the counselee account ID as destination information through the destination input interface, and based thereon, may send medical consultation content that does not include counselee information to the specific counselee terminal 100-2.

In this connection, the counselor terminal 100-1 may provide the counselee terminal 100-2 with the corresponding online printout either through texting or a medical consultation application method.

In detail, in the case of providing an online printout through a medical consultation application, the counselor terminal 100-1 sends the medical consultation content to the medical consultation server 200 by matching it with the service account of the counselee entered through the destination input interface, so that the medical consultation server 200 may immediately match and store the service account and medical consultation content. Accordingly, the counselor terminal 100-1 may also convert the medical consultation content that does not include counselee information into medical consultation content including counselee information and then store the same.

In addition, when the counselor terminal 100-1 sends an online printout to the counselee terminal 100-2 using texting, the corresponding online printout, that is, the medical consultation content that does not include counselee information, is sent to the specific counselee terminal 100-2, based on the terminal identification number acquired through the destination input interface.

In this connection, when the medical consultation content is sent using texting, the counselor terminal 100-1 may generate a link to download the corresponding medical consultation content, and may include the generated link in the corresponding text message and transmit the same.

Thus, the counselor terminal 100-1 may output the medical consultation content matched with the corresponding link as a graphic image when a selection input for the link of the text message received from the counselee terminal 100-2 is detected.

When the corresponding online printout is sent to the counselee terminal 100-2 in the form of a medical consultation application, based on the counselee account ID and/or terminal identification number acquired through the destination input interface, the counselor terminal 100-1 may send the corresponding online printout, that is, the medical consultation content that does not include counselee information, to the specific counselee terminal 100-2. In an embodiment, when the counselor terminal 100-1 transmits the medical consultation content to the medical consultation server 200 by matching the account ID of a counselee, the medical consultation server 200 sends medical consultation content to the counselee terminal 100-2, which is matched with the account information of the counselee. In this connection, the counselee terminal 100-2 may transmit the medical consultation content in different ways depending on whether the medical consultation application is installed. In addition, when the counselee account information and medical consultation content are transmitted together from the counselor terminal 100-1, the medical consultation server 200 may match the same and store the same in a database, and then provide the same when a counselor/consultee requests to view medical consultation content for an account.

When a medical consultation application is presently installed in the specific counselee terminal 100-2, the medical consultation server 200 may output medical consultation content through the medical consultation application installed in the corresponding counselee terminal 100-2.

When the medical consultation application is not installed in the specific counselee terminal 100-2, the medical consultation server 200 may provide a medical consultation application installation process capable of installing the medical consultation application on the corresponding counselee terminal 100-2.

For example, the medical consultation server 200 may first determine whether a medical consultation application is installed in the corresponding counselee terminal 100-2. In addition, when it is determined that the medical consultation application is not installed, the counselee terminal 100-1 may forward a text message including a link to a medical consultation application installation page to the corresponding counselee terminal 100-2 based on the terminal identification number of the corresponding counselee. Thereafter, when a selection input for the link of the text message received from the counselee terminal 100-2 is detected, an installation process may be provided by moving to the medical consultation application installation page.

In addition, the medical consultation server 200 may output the corresponding medical consultation content matched with the counselee account through the installed medical consultation application when installation of the medical consultation application is completed in the counselee terminal 100-2. A detailed description thereof will be described below.

Next, based on the destination information input through the destination input interface, the counselor terminal 100-1 that outputs the medical consultation content that does not include the counselee information to the specific counselee terminal 100-2 may acquire the counselee information for the output medical consultation content (S123).

In detail, in an embodiment of the present disclosure, the counselor terminal 100-1 may interlock with the counselee terminal 100-2 and/or the medical consultation server 200 to acquire counselee information for medical consultation content that does not include counselee information.

In more detail, based on the counselee information pre-stored on the counselee terminal 100-2 that has transmitted the medical consultation content, the counselor terminal 100-1 may store the medical consultation content that does not include counselee information as medical consultation content matched with counselee information.

First, the counselee terminal 100-2 that has received the medical consultation content that does not include counselee information from the counselor terminal 100-1 may output the received medical consultation content through the display unit 130.

In this connection, when the counselee terminal 100-2 outputs medical consultation content that does not include counselee information in conjunction with the installed medical consultation application, the service account information generated and pre-stored when signing up for the corresponding medical consultation application may be matched when the corresponding medical consultation application is driven.

Further, the counselee terminal 100-2 may send the matched service account information (consultee information) to the medical consultation server 200 when outputting the received medical consultation content.

Subsequently, the medical consultation server 200 that has received the medical consultation content and the service account information (counselee information) matched with the corresponding medical consultation content from the counselee terminal 100-2 may store the received medical consultation content and matched service account information (counselee information) in the database 230.

Further, the medical consultation server 200 may provide the account information of a counselee matched with the medical consultation content to the counselor terminal 100-1 and provide the medical consultation content including the counselee information.

As a result, the counselee terminal 100-1 may acquire counselee information for the output medical consultation content that does not include the counselee information through interlocking with the counselee terminal 100-2 and/or the medical consultation server 200.

Further, the counselor terminal 100-1 may convert the medical consultation content that does not include the counselee information into medical consultation content including the counselee information and store the same by matching and storing the counselee information for the acquired medical counselee content that does not include the counselee information with the corresponding medical consultation content.

In other words, in this way, the counselor terminal 100-1 may convert the medical consultation content that does not include the counselee information into medical consultation content including the counselee information based on the counselee information pre-stored on the counselee terminal 100-2 that has transmitted the medical consultation content. Hence, an owner may decide a counselee matching the undecided medical consultation content, and enable systematic management by tying the decided counselee and the corresponding medical consultation content together.

In addition, the counselor terminal 100-1 may extract and provide medical consultation content matched with the counselee information based on the stored medical consultation content matched for each counselee information when the corresponding counselee visits again later.

In an embodiment of the present disclosure, it is described that the counselee terminal 100-2 transmits medical consultation content and service account information (counselee information) matched with the corresponding medical consultation content to the medical consultation server 200, and transmits the medical consultation content and the service account information (counselee information) matched with the corresponding medical consultation content from the medical consultation server 200 to the counselor terminal 100-1. However, various embodiments may be possible, such as direct transmission of the medical consultation content and service account information (counselee information) matched with the corresponding medical consultation content from the counselee terminal 100-2 to the counselor terminal 100-1.

In addition, in an embodiment of the present disclosure, the counselee terminal 100-1 may acquire counselee information based on the counselee information input interface through which the counselee information may be input on the counselee terminal 100-2. A detailed description thereof will be described later.

When the medical consultation content that does not include counselee information is provided as an offline printout, the counselor terminal 100-1 may transfer the medical consultation content that does not include counselee information to an external output device (for example, printer) to provide the offline printout (S125).

In this connection, the counselor terminal 100-1 may display and output an offline printout of medical consultation content output through an external output device together with a predetermined identification code 1 (for example, a QR code) matching the corresponding medical consultation content.

Herein, when the terminal 100 detects the identification code 1 displayed on the offline printout through the camera 150, the identification code 1 may provide an interface for requesting input of counselee information or provide medical consultation installed in the terminal 100, execute a medical consultation application installed in the corresponding terminal 100 to output medical consultation content matching the corresponding identification code 1, or provide a medical consultation application installation process when the medical consultation application is not installed in the corresponding terminal 100.

In an embodiment, the counselee terminal 100-2 may recognize the identification code 1 to store an offline printout as an online printout, and may install a medical consultation application for management convenience.

In detail, the counselee terminal 100-2 may recognize the identification code 1 to execute the medical consultation application installation process. After installing the medical consultation application, when the counselee information is input and a member is signed up and then logged in, the medical consultation content matched with the identification code 1 may be matched with the counselee information (service account). In other words, after storing the medical consultation content that does not include the counselee information matched with the identification code 1 from the counselor terminal 100-1, and then receiving the information recognizing the identification code 1 in a specific counselee service account, the medical consultation server 200 may provide a counselee with medical consultation content matched with the corresponding identification code 1 through the counselee service account. In addition, by matching the counselee service account and storing the medical consultation content, the counselor terminal 100-1 may also match the medical consultation content with the counselee information and receive the information so as to be viewed again during subsequent consultation.

Accordingly, the counselor terminal 100-1 may acquire counselee information matched with medical consultation content that does not include counselee information, and may generate and store medical consultation content including the counselee information (S127).

In other words, the counselor terminal 100-1 acquires counselee information matched with medical consultation content whose owner is unknown, and sends the medical consultation content matched with the acquired counselee information to the medical consultation server 200, thereby effectively assisting in the construction of the medical consultation content management database 230 for each counselee in the medical consultation server 200.

Method for Managing Medical Consultation Content for Single Counselee in Counselee Terminal

Hereinafter, a method for managing medical consultation content when a single counselee is signed up for a medical consultation application in the counselee terminal 100-2 according to an embodiment of the present disclosure will be described in detail. However, overlapping descriptions of the contents described above may be summarized or omitted.

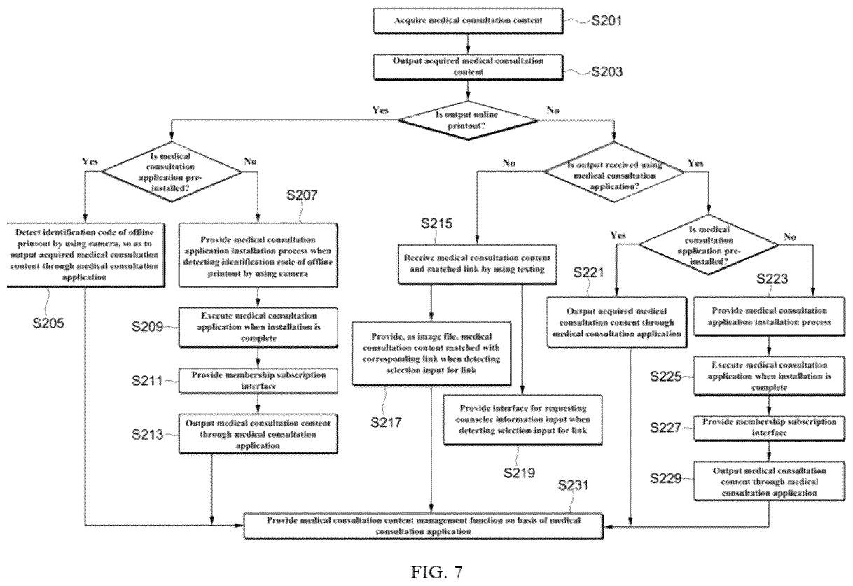

FIG. 7 is a flowchart illustrating a method for managing medical consultation content when a single counselee exists in the counselee terminal 100-2 according to an embodiment of the present disclosure.

Referring to FIG. 7, in the embodiment, the counselee terminal 100-2 may first acquire medical consultation content (S201).

In detail, the counselee terminal 100-2 may receive medical consultation content related to the corresponding counselee from the counselee terminal 100-1.

In this connection, the counselee terminal 100-2 may acquire the medical consultation content as an offline printout and/or an online printout.

In an embodiment, the counselee terminal 100-2 may acquire medical consultation content as data based on an offline printout including a predetermined identification code 1.

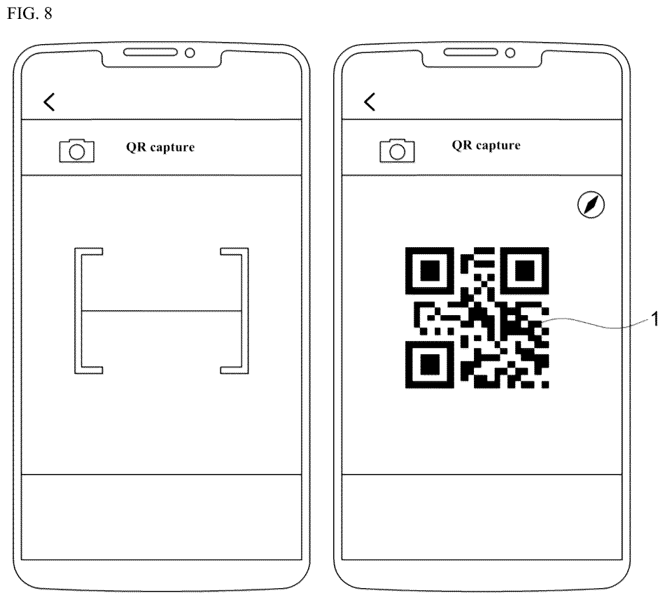

FIG. 8 is a diagram for explaining a method for acquiring medical consultation content on the basis of the identification code 1 of an offline printout according to an embodiment of the present disclosure.

In detail, referring to FIG. 8, the counselee terminal 100-2 may acquire medical consultation content based on the predetermined identification code 1 (for example, QR code) included in an offline printout of the medical consultation content output through an external output device.

In more detail, first, the counselee terminal 100-2 may detect/sense the identification code 1 of the offline printout using the camera 150.

In addition, when the counselee terminal 100-2 detects the identification code 1 based on the camera 150, it is connected to the medical consultation server 200 and may receive medical consultation content matched with the corresponding identification code 1.

In this connection, when there is no counselee information that matches the received medical consultation content, the counselee terminal 100-2 may provide a counselee information input interface requesting input of the counselee information when the identification code 1 of the offline printout is detected through the camera 150.

In detail, the counselee terminal 100-2 may first check whether to agree to the provision of the counselee information input through the counselee information input interface.

In an embodiment, the counselee terminal 100-2 may be connected to a link for install a medical consultation application upon detecting the identification code 1, and provide an interface for providing counselee information for signing up as a member/login after installing the medical consultation application.

Further, the counselee terminal 100-2 may send counselee information input through a counselee information providing interface to the medical consultation server 200.

The medical consultation server 200 may match the medical consultation content matched with the identification code 1 received from the counselor terminal 100-1 with the counselee information, and then provide the medical consultation content to the counselee terminal 100-2.

In another embodiment, the counselee terminal 100-2 may check whether to agree to the provision of counselee information input through a counselee information input interface provided through a web browser application.

Herein, the provision of counselee information may include provision of counselee information to the medical consultation server 200 and/or the counselee terminal 100-1.

Further, when the provision of the counselee information is accepted, the counselee terminal 100-2 may provide a counselee information input interface.

In addition, the counselee terminal 100-2 may match the counselee information acquired through the provided counselee information input interface with the received medical consultation content and store them in the memory 160.

In addition, the counselee terminal 100-2 may send counselee information and medical consultation content matched with the corresponding counselee information to the medical consultation server 200 and/or the counselee terminal 100-1.

Thus, also as for the medical consultation content transferred to a counselee through the offline printout, the counselee terminal 100-2 may clarify the owner matched with the corresponding medical consultation content, store the same in the database 230, and manage the same.

In contrast, when provision of counselee information is rejected, the counselee terminal 100-2 may manage the received medical consultation content as medical consultation content whose owner does not exist.

In other words, when the provision of counselee information is rejected, the medical consultation content received through the counselee terminal 100-2 may remain as owner-nonexistent medical consultation content without matching counselee information.

In addition, in this connection, the counselee terminal 100-2 may not provide a counselee information input interface, and may transmit the medical consultation content matched with the corresponding identification code 1 detected through the camera 150 to data such as an image file.

The counselee terminal 100-2 may acquire medical consultation content based on the online printout received from the counselor terminal 100-1.

In detail, the counselee terminal 100-2 may receive medical consultation content from the counselor terminal 100-1 either through texting or a medical consultation application method.

In this connection, when there is no counselee information matching the corresponding medical consultation content, the counselee terminal 100-2 may receive the medical consultation content based on the destination input interface provided by the counselee terminal 100-1.

In an embodiment, the counselor terminal 100-1 may acquire a terminal identification number and/or a counselee account ID (for example, SNS ID) as destination information through a destination input interface, and based thereon, may transmit medical consultation content that does not include counselee information to the specific counselee terminal 100-2.

Herein, when a medical consultation application is presently installed in the corresponding counselee terminal 100-2, the counselee terminal 100-2 may interlock with the corresponding medical consultation application, and may match and store pre-stored counselee information with the received medical consultation content.

In addition, the counselee terminal 100-2 may send the matched and stored counselee information and medical consultation content to the medical consultation server 200 and/or the counselee terminal 100-1.

When the counselee terminal 100-2 does not have a medical consultation application installed or receives medical consultation content using texting, the corresponding counselee terminal 100-2 may provide a counselee information input interface that requests input of counselee information.

In detail, the counselee terminal 100-2 may first check whether to agree to the provision of the counselee information input through the counselee information input interface. When the provision of the counselee information is accepted, it may provide the counselee information input interface.

In addition, the counselee terminal 100-2 may match the counselee information acquired through the provided counselee information input interface with the received medical consultation content and store them in the memory 160.

In addition, the counselee terminal 100-2 may transmit counselee information and medical consultation content matched with the corresponding counselee information to the medical consultation server 200 and/or the counselee terminal 100-1.

In contrast, when provision of counselee information is rejected, the counselee terminal 100-2 may manage the received medical consultation content as medical consultation content whose owner does not exist.

In addition, in this connection, the counselee terminal 100-2 may not provide a counselee information input interface, and may directly perform a function of providing medical consultation content in the form of an image file (for example, providing medical consultation content based on a link of a text message).

Next, the counselee terminal 100-2 that has acquired the medical consultation content may output the acquired medical consultation content (S203).

In detail, the counselee terminal 100-2 may output, through the display unit 130, the medical consultation content acquired based on the offline printout or the online printout.

First, when the medical consultation content is acquired as an offline printout, the counselee terminal 100-2 may output the medical consultation content depending on whether a medical consultation application is installed in the corresponding counselee terminal 100-2.

In detail, when the medical consultation application is presently installed in the counselee terminal 100-2 at the time of outputting medical consultation content acquired through an offline printout, the corresponding counselee terminal 100-2 may output the medical consultation content through the corresponding medical consultation application (S205).

In other words, the counselee terminal 100-2 may output the medical consultation content acquired by detecting the identification code 1 of the offline printout using the camera 150 by driving a medical consultation application installed in the corresponding counselee terminal 100-2.

In contrast, when the medical consultation application is not installed in the counselee terminal 100-2 at the time of outputting the medical consultation content acquired through the offline printout, the counselee terminal 100-2 may provide a medical consultation application installation process when detecting the identification code 1 of the offline printout through the camera 150 (S207).

Herein, in an embodiment of the present disclosure, when the medical consultation application is not installed on the terminal 100 at the time when each piece of medical consultation content is identified and detected through the camera 150 of the corresponding terminal 100 at the same time, the identification code 1 may play a role of providing a medical consultation application installation process.

In detail, when the medical consultation application is not installed on the counselee terminal 100-2 at the time of acquiring medical consultation content through the identification code 1, the corresponding counselee terminal 100-2 may move to an installation page that provides a process of installing the medical consultation application based on the corresponding identification code 1 and download and install the medical consultation application.

When the download and installation of the medical consultation application are completed, the counselee terminal 100-2 may execute the corresponding medical consultation application and provide a member registration interface (S209 and S211).

In this connection, the counselee terminal 100-2 may generate service account information including counselee information of the corresponding counselee through the member registration interface for the medical consultation application.

For example, the counselee terminal 100-2 may acquire account information including an account ID, name, gender, age, resident registration number, and/or terminal identification number (phone number) of a counselee based on the member registration interface.

Thereafter, the counselee terminal 100-2 may output and provide medical consultation content acquired based on the identification code 1 of the offline printout through the corresponding medical consultation application (S213).

For example, upon completion of installation of a medical consultation application, the counselee terminal 100-2 may automatically execute the corresponding application to output the acquired medical consultation content.

When acquiring the medical consultation content as an online printout, the counselee terminal 100-2 may output the medical consultation content depending on whether the online printout is received based on a medical consultation application.

In detail, when the counselee terminal 100-2 does not receive the online printout based on the medical consultation application, it may acquire the corresponding online printout (medical consultation content) using texting.

In detail, the counselee terminal 100-2 may receive a text message including a link capable of providing medical consultation content acquired as an online printout in the form of a graphic image (S215).

In this connection, the counselor terminal 100-1 that sends the medical consultation content to the counselee terminal 100-2 using texting may generate a link to provide the corresponding medical consultation content as a graphic image, and transmit the generated link by including the same in the corresponding text message.

Thereafter, the counselee terminal 100-2 may output the medical consultation content matched with the corresponding link as a graphic image when a selection input for the link of the text message received is detected (S217).

For example, upon detecting input of a link included in a text message, the counselee terminal 100-2 may display the medical consultation content by automatically driving a predetermined application (for example, a file reader application) that displays the corresponding medical consultation content matched with the corresponding link as a graphic image.

In this connection, the counselee terminal 100-2 may provide a counselee information input interface requesting the counselee information input of the corresponding counselee upon detecting a selection input for a link (S219).

In detail, the counselee terminal 100-2 may first check whether to agree to the provision of the counselee information input through the counselee information input interface, and when the provision of the counselee information is accepted, it may provide the counselee information input interface.

In addition, the counselee terminal 100-2 may match the counselee information acquired through the provided counselee information input interface with the received medical consultation content and store them in the memory 160.

In addition, the counselee terminal 100-2 may transmit counselee information and medical consultation content matched with the corresponding counselee information to the medical consultation server 200 and/or the counselee terminal 100-1.

In contrast, when provision of counselee information is rejected, the counselee terminal 100-2 may manage the received medical consultation content as medical consultation content whose owner does not exist.

In addition, in this connection, the counselee terminal 100-2 may not provide a counselee information input interface, and may directly perform a function of providing medical consultation content in the form of an image file.

When receiving an online printout based on a medical consultation application, the counselee terminal 100-2 may output medical consultation content depending on whether the medical consultation application is installed in the corresponding counselee terminal 100-2.

First, when the counselee terminal 100-2 acquires an online printout based on a medical consultation application, the corresponding online printout (medical consultation content) may be output through the medical consultation application in the case where the medical consultation application is presently installed in the corresponding counselee terminal 100-2 (S221).

In other words, the counselee terminal 100-2 may output the acquired medical consultation content by driving the medical consultation application installed in the corresponding counselee terminal 100-2.

When acquiring an online printout based on the medical consultation application, the counselee terminal 100-2 may be provided with a medical consultation application installation process in the case where the medical consultation application is not installed in the corresponding counselee terminal 100-2 (S223).

In detail, when the counselee terminal 100-2 acquires the medical consultation content as an online printout but the medical consultation application is not installed in the corresponding counselee terminal 100-2, a medical consultation application installation process may be provided from the counselor terminal 100-1.

In detail, the counselor terminal 100-1 that has transmitted the online printout to the counselee terminal 100-2 may determine whether a medical consultation application is installed in the corresponding counselee terminal 100-2.

In addition, when it is determined that the medical consultation application is not installed, the counselee terminal 100-1 may forward a text message including a link to a medical consultation application installation page to the corresponding counselee terminal 100-2 based on the terminal identification number of the corresponding counselee.

Thereafter, when a selection input for the link of the text message received from the counselee terminal 100-2 is detected, an installation process may be provided by moving to the medical consultation application installation page.

In addition, the counselee terminal 100-2 may download and install the medical consultation application by executing the provided medical consultation application installation process.

In an embodiment of the present disclosure, it is described that a medical consultation application installation process is provided from the counselor terminal 100-1 to the counselee terminal 100-2. However, in another embodiment, when the medical consultation application is not installed in the counselee terminal 100-2 when receiving medical consultation content, the corresponding counselee terminal 100-2 may autonomously provide the medical consultation application installation process.

When the download and installation of the medical consultation application are completed, the counselee terminal 100-2 may execute the corresponding medical consultation application and provide a member registration interface (S225 and S227).

In this connection, the counselee terminal 100-2 may generate service account information including counselee information of the corresponding counselee through the member registration interface for the medical consultation application.

For example, the counselee terminal 100-2 may acquire account information including an account ID, name, gender, age, resident registration number, and/or terminal identification number (phone number) of a counselee based on the member registration interface.

Thereafter, the counselee terminal 100-2 may output and provide the acquired medical consultation content as an online printout through the corresponding medical consultation application (S229).

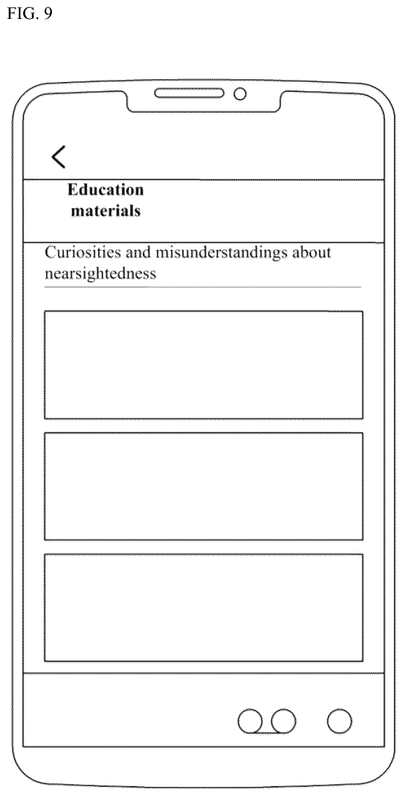

FIG. 9 is an example showing a process of outputting medical consultation content as an online printout according to an embodiment of the present disclosure.

Referring to FIG. 9, for example, when installation of a medical consultation application is completed, the counselee terminal 100-2 may automatically execute the corresponding application and output the obtained medical consultation content as shown in FIG. 9.

As such, in an embodiment of the present disclosure, the counselee terminal 100-2 may acquire medical consultation content as an offline printout and/or an online printout, and receive and output the medical consultation content in a manner optimized for each printout. Thereby, the efficiency of sharing and managing the medical consultation content can be increased.

In addition, when there is no counselee information matching the acquired medical consultation content, the counselee terminal 100-2 according to an embodiment of the present disclosure may acquire counselee information for the corresponding medical consultation content, and manage medical consultation content matched with the acquired counselee information as well. Thereby, it is possible to manage medical consultation content more systematically by classifying the medical consultation content by counselee.

In addition, the counselee terminal 100-2 may provide a medical consultation content management function based on a medical consultation application (S231).

In detail, the counselee terminal 100-2 may manage at least one piece of the received medical consultation content through a medical consultation application installed in the corresponding counselee terminal 100-2.

Herein, the medical consultation content management function may include classification management, deletion management, and/or counselee information interlocking management function for at least one piece of medical consultation content.

In other words, the counselee terminal 100-2 may provide a medical consultation content management function that comprehensively manages medical consultation content interlocked to the counselee information.

For example, the counselee terminal 100-2 may provide a function of classifying and checking the medical consultation content of a counselee by date, medical institution, and/or medical treatment subject through a medical consultation application, a function of deleting the medical consultation content, and/or a function of setting linkage of counselee information for each piece of medical consultation content.

In this connection, the interlocking management of counselee information may include a function of deciding whether to interlock the counselee information with respect to each piece of medical consultation content existing in the medical consultation application according to the selection of a counselee.

In other words, in consideration of the case where the corresponding counselee wants to manage his/her counselee information in a non-linked manner with specific medical consultation content (for example, medical consultation content related to urology consultation, medical consultation content related to obstetrics and gynecology consultation, etc., which are closely related to personal privacy), it is possible to provide a counselee information interlocking management function capable of setting whether to interlock counselee information for each piece of medical consultation content.

For example, according to the selection of a counselee, first medical consultation content of the medical consultation application may be set to be managed in conjunction with the counselee information, and the second medical consultation content may be set to be managed in a non-interlocked manner with the counselee information. In other words, the second medical consultation content may be set to be managed as non-owner medical consultation content managed without matched counselee information.

As such, in an embodiment of the present disclosure, the counselee terminal 100-2 provides a function of managing the medical consultation content of a counselee based on the medical consultation application, so that the medical consultation content for at least one counselee can be checked and managed systematically. Furthermore, it is possible to assist a counselee to easily utilize his or her own medical treatment information.

In addition, when the counselee terminal 100-1 requests to view the medical consultation content according to the counselee information, the counselee terminal 100-2 may approve/disapprove the output from the counselor terminal 100-1 for the interlocked medical consultation content.

In detail, when there is a request to view medical consultation content matched to the counselee information from the counselor terminal 100-1, it may be possible to interlock with the medical consultation server 200, and receive a message approving or disapproving whether to view the medical consultation content according to a viewing request. According to a response to the message, the counselor terminal 100-1 may determine whether to view the medical consultation content.

Method for Managing Medical Consultation Content for Plurality of Counselees in Counselee Terminal

Hereinafter, a method for managing medical consultation content when a plurality of counselees are registered in a medical consultation application in the counselee terminal 100-2 according to an embodiment of the present disclosure will be described in detail. However, overlapping descriptions of the contents described above may be summarized or omitted.

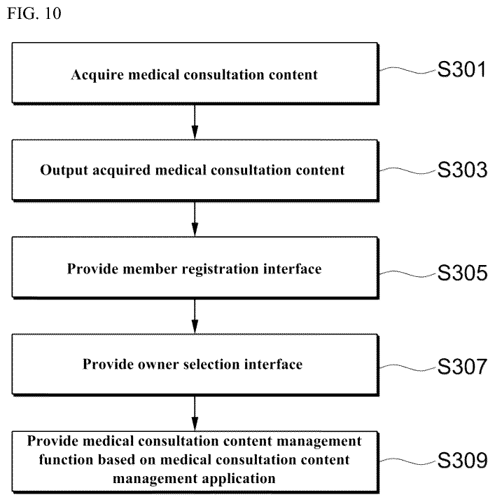

FIG. 10 is a flowchart illustrating a method for managing medical consultation content when a plurality of counselees exist in the counselee terminal 100-2 according to an embodiment of the present disclosure.

Referring to FIG. 10, in an embodiment, the counselee terminal 100-2 may first acquire medical consultation content (S301).

In detail, the counselee terminal 100-2 may receive medical consultation content related to the corresponding counselee from the counselee terminal 100-1.

In this connection, the counselee terminal 100-2 may acquire the medical consultation content as an offline printout and/or an online printout.

In addition, the counselee terminal 100-2 may output the acquired medical consultation content (S303).

In detail, the counselee terminal 100-2 may output the medical consultation content acquired based on an offline printout or an online printout through the display unit 130.

More specifically, when the medical consultation content is acquired as an offline printout, the counselee terminal 100-2 may output the medical consultation content depending on whether a medical consultation application is installed in the corresponding counselee terminal 100-2.

In this connection, the counselee terminal 100-2 may output the acquired medical consultation content through the corresponding medical consultation application when a medical consultation application is presently installed in the corresponding counselee terminal 100-2.

Conversely, when the corresponding counselee terminal 100-2 does not have the medical consultation application installed in the counselee terminal 100-2, the counselee terminal 100-2 may provide a medical consultation application installation process when detecting the identification code 1 of the offline printout through the camera 150.

In detail, when the medical consultation application is not installed on the counselee terminal 100-2 at the time of acquiring medical consultation content through the identification code 1, the corresponding counselee terminal 100-2 may move to an installation page that provides a process of installing the medical consultation application based on the corresponding identification code 1 and download and install the medical consultation application.

When acquiring the medical consultation content as an online printout, the counselee terminal 100-2 may output the medical consultation content depending on whether the online printout is received based on a medical consultation application.

In detail, when the counselee terminal 100-2 does not receive the online printout based on the medical consultation application, it may acquire the corresponding online printout (medical consultation content) using texting.

In detail, the counselee terminal 100-2 may receive a text message including a link capable of providing medical consultation content acquired as an online printout in the form of a graphic image, and may output the medical consultation content matched with the corresponding link as a graphic image when a selection input for the link of the text message received is detected.

When receiving an online printout based on a medical consultation application, the counselee terminal 100-2 may output medical consultation content depending on whether the medical consultation application is installed in the corresponding counselee terminal 100-2.

First, when the counselee terminal 100-2 acquires an online printout based on a medical consultation application, the corresponding online printout (medical consultation content) may be output through the medical consultation application in the case where the medical consultation application is presently installed in the corresponding counselee terminal 100-2.

Conversely, when acquiring an online printout based on the medical consultation application, the counselee terminal 100-2 may be provided with a medical consultation application installation process in the case where the medical consultation application is not installed in the corresponding counselee terminal 100-2.

In addition, the counselee terminal 100-2 may download and install the medical consultation application by executing the provided medical consultation application installation process.

In addition, when the download and installation of the medical consultation application are completed, the counselee terminal 100-2 may execute the corresponding medical consultation application and provide a member registration interface.

In this connection, the counselee terminal 100-2 may generate service account information including counselee information of the corresponding counselee through the member registration interface for the medical consultation application.

For example, the counselee terminal 100-2 may acquire account information including an account ID, name, gender, age, resident registration number, and/or terminal identification number (phone number) of a counselee based on the member registration interface.

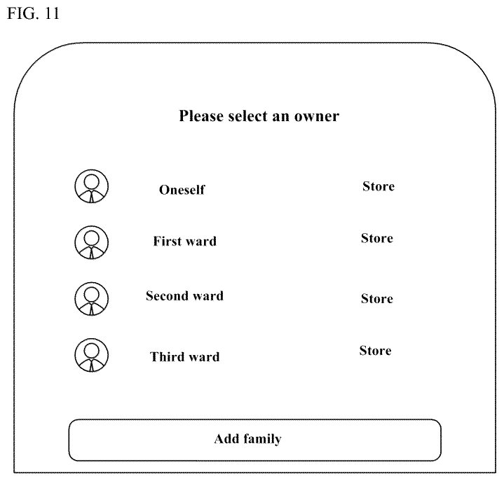

FIG. 11 is an example of a member registration interface according to an embodiment of the present disclosure.

In addition, referring to FIG. 11, in an embodiment of the present disclosure, the counselee terminal 100-2 may further provide a member registration interface based on a medical consultation application (S305).

Herein, the member registration interface may be an interface through which a plurality of counselees may be registered by adding at least one counselee other than the corresponding counselee as members to the medical consultation application installed in the counselee terminal 100-2.

In general, when performing medical consultation, there may be a case in which a counselee alone may have difficulty in managing the provided medical consultation content, such as when the counselee is a minor or an elderly person. In this connection, it is necessary for a guardian of the corresponding counselee to take the place of the corresponding counselee.

Accordingly, in an embodiment of the present disclosure, it is intended to provide a medical consultation content management service in which a counselee may manage medical consultation content of another counselee as a guardian of the other counselee.

In other words, in an embodiment of the present disclosure, the counselee terminal 100-2 may provide a member registration interface through which a counselee may register at least one other counselee as a member in his/her medical consultation application, and may provide a function of managing medical consultation content for at least one registered member through the member registration interface.

In detail, the counselee terminal 100-2 may first acquire counselee information about at least one other counselee based on a member registration interface.

Further, the counselee terminal 100-2 may add and register members in the corresponding medical consultation application based on the acquired counselee information about other counselees.

For example, the counselee terminal 100-2 may acquire at least one of a name, gender, age, resident registration number, and/or terminal identification number (telephone number) of another counselee through the member registration interface, and may perform member registration by generating member information included in the medical consultation application based on the acquired information.

For example, the counselee terminal 100-2 may register a first counselee (for example, the counselee himself or herself), a second counselee (for example, one of the counselee's children), and a third counselee (for example, one of the counselee's parents) as members in a medical consultation application installed in the corresponding counselee terminal 100-2.

In an embodiment, in order to secure the reliability of member registration and to provide a process for members to directly manage their own medical consultation content later, the counselee terminal 100-2 may go through a process of verifying a legal identity when registering additional counselees as members.

For example, the counselee terminal 100-2 may 1) perform a member authentication procedure through a procedure of registering official documents such as a family relationship confirmation certificate or a legal guardian confirmation as an image file, 2) perform a confirmation procedure by sending a message to approve member registration for a counselee from the terminal of a member himself/herself to a member terminal, and 3) perform a procedure for confirming that a member is a ward of a counselee by directly interlocking with a public institution server.

Thereafter, the counselee terminal 100-2 may provide a function of receiving and managing medical consultation content for at least one member registered through the member registration interface from the counselor terminal 100-1.

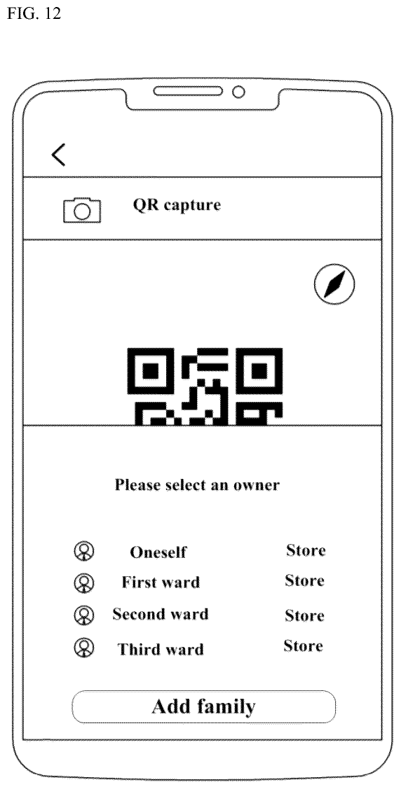

FIG. 12 is an example of an owner selection interface according to an embodiment of the present disclosure.

In addition, in an embodiment of the present disclosure, the counselee terminal 100-2 may further provide an owner selection interface based on a medical consultation application (S307).

In detail, when receiving medical consultation content through the corresponding medical consultation application after a plurality of counselees are registered as members in the medical consultation application, the counselee terminal 100-2 may provide an owner selection interface capable of matching the received medical consultation content with any one of at least one member.

In other words, the counselee terminal 100-2 may match and store the received medical consultation content with the counselee information of the selected member based on the owner selection interface.

In addition, the counselee terminal 100-2 may output medical consultation content matched with the counselee information of the selected member through the display unit 130.

As such, in an embodiment of the present disclosure, the counselee terminal 100-2 may register a plurality of counselees as members in the medical consultation application, and may decide counselee information to be matched with the received medical consultation content based on the counselee information of registered members. In addition, as the counselee terminal 100-2 matches, manages, and provides the decided counselee information with the received medical consultation content, the counselee terminal 100-2 may allow the counselee to act as an agent as a guardian by managing medical consultation content for other counselees in addition to the counselee, and may easily and systematically manage medical consultation content for multiple counselees.

In addition, the counselee information of members decided in the counselee terminal 100-2 as such may be transmitted to the medical consultation server 200, matched with the medical consultation content, and stored.

Accordingly, it is possible to check the medical consultation content matched with the counselee information input when the counselee information is input at another terminal thereafter. For example, when the medical consultation content of a first guardian registered in the terminal of the first guardian is authenticated (personal authentication or guardian authentication) after inputting the first guardian information in the second guardian's terminal or counselor's terminal, it is possible to view the medical consultation content.

In addition, the counselee terminal 100-2 may provide a medical consultation content management function based on a medical consultation application (S309).

In detail, the counselee terminal 100-2 may manage at least one piece of the received medical consultation content through a medical consultation application installed in the corresponding counselee terminal 100-2.

In this connection, the counselee terminal 100-2 may check and manage medical consultation content matched by at least one piece of the counselee information registered in the medical consultation application (by member).

Herein, the medical consultation content management function may include classification management, deletion management, and/or counselee information interlock management functions for the at least one piece of medical consultation content.

In particular, in an embodiment, the counselee information interlock management may include: 1) a function of determining whether counselee information is interlocked with each piece of medical consultation content existing in the medical consultation application according to the selection of a counselee, and may further include 2) a function of selecting counselee information (member information) to be interlocked with respect to each piece of medical consultation content existing in the medical consultation application in the case where there are two or more counselees registered in the medical consultation application.

For example, the counselee terminal 100-2 may set the first medical consultation content of the medical consultation application to be managed in conjunction with the first counselee information according to the selection of a counselee based on the counselee information interlock management, and may set the second medical consultation content to be managed in conjunction with the second counselee information.

In addition, the counselee terminal 100-2 may transmit the acquired medical consultation content to other guardians. In detail, the counselee terminal 100-2 may grant the authority to manage the medical consultation content to the second guardian or guardian's terminal. In this connection, the medical consultation server 200 may proceed with an authentication process to view and manage the medical consultation content after receiving guardian authentication, personal authentication, or personal viewing approval certification.

In addition, the counselee terminal 100-2 that has received guardian authentication may check and manage medical consultation content stored in a list matched with members who are wards.

As such, in an embodiment of the present disclosure, the counselee terminal 100-2 may manage medical consultation content for a plurality of counselees based on a medical consultation application, thereby expanding the scope of management of the medical consultation content. In addition, it is possible to increase the effectiveness of managing a plurality of pieces of medical consultation content.

A ward member terminal may want the medical consultation content not to be exposed to the ward counselee terminal 100-2 in order to protect the privacy of a guardian.

To this end, the counselor terminal 100-1 may include information related to non-interlocking guardians when issuing an online/offline printout. In detail, the counselor terminal 100-1 may activate the guardian non-interlocking function before issuing printouts when privacy protection is required even for the guardian or when there is a request for a member.

When the guardian non-interlocking function is activated, the code for the guardian non-interlocking function is inserted into the identification code 1 of the printout, and there may be provided such that only the terminal recognizing the identification code 1 may check the medical consultation content.

In addition, when the guardian non-interlocking function is activated, the counselor terminal 100-1 may transmit the non-interlocking of the guardian of the medical consultation content for the identification code 1 to the medical consultation server 200. Later, when the medical consultation server 200 sets the medical consultation content counselee information for the identification code 1, the medical consultation content may be viewed and managed only by the terminal that has scanned the identification code 1.

In addition, the medical consultation server 200 may set the non-interlocked medical consultation content not to be exposed on the guardian's terminal of a counselee.

As described above, the method and system for managing medical consultation content according to an embodiment of the present disclosure enable extension of the available range of patient medical treatment information by safely and easily sharing and managing medical treatment information of patients.

In addition, the method and system for managing medical consultation content according to an embodiment of the present disclosure enable patients to acquire and manage their own medical treatment information, so that not only hospitals but also patients themselves are able to check and manage their own medical treatment information. Also, when a patient is transferred to another hospital, the convenience of exchanging medical treatment information for patients can be increased.

In addition, the method and system for managing medical consultation content according to an embodiment of the present disclosure enable a guardian of an authenticated patient to acquire and manage the medical treatment information of the patient, so that when the guardian needs to manage the patient, such as when the patient is a minor, the guardian of the patient is able to check and manage the medical treatment information of the patient, and thus, more effective patient care can be assisted.

In addition, the method and system for managing medical consultation content according to an embodiment of the present disclosure generate and provide medical treatment information of a patient that can replace the EMR, which is medical treatment information that is difficult to share with the private sector under the current medical law. Accordingly, accessibility to the medical treatment information of the patient can be improved, and utilization of patient medical treatment information can be enhanced.

In addition, the method and system for managing medical consultation content according to an embodiment of the present disclosure generate and provide medical treatment information of a patient that can replace the EMR, thus enabling the use of base data that may assist patients' treatment in a more diversified way other than the EMR formed in a standardized manner in medical institutions where patients are treated.

In addition, embodiments of the present disclosure described above may be implemented in the type of program commands the can be executed through various computer components, and may be recorded on a computer-readable recording medium. The computer-readable recording medium may include program commands, data files, and data structures individually or in combinations thereof. The program commands that are recorded on a computer-readable recording medium may be those specifically designed and configured for the present disclosure or may be those available and known to those engaged in computer software in the art. The computer-readable recording medium includes magnetic media such as hard disks, floppy disks, and magnetic media such as a magnetic tape, optical media such as CD-ROMs and DVDs, magneto-optical media such as floptical disks, and hardware devices specifically configured to store and execute program commands, such as ROM, RAM, and flash memory. The program commands include not only machine language codes compiled by a compiler, but also high-level language code that can be executed by a computer using an interpreter etc. A hardware device may be changed into one or more software module to perform the processes according to the present disclosure, and vice versa.

What is claimed is:

1. A method for managing medical consultation content of a counselee performed in a processor of a counselee terminal, the method including:
acquiring the medical consultation content of the counselee through at least one medium of an offline printout or an online printout;
acquiring counselee information matched with the medical consultation content when the counselee information matched with the acquired medical consultation content does not exist;
matching the acquired counselee information with the medical consultation content and storing the same;
controlling a display to output the medical consultation content matched with the counselee information; and
managing the stored medical consultation content on the basis of a medical consultation application.

2. The method of claim 1, wherein the counselee information is information for deciding a counselee identification and an owner of the medical consultation content, and includes at least one of a counselee name, gender, age, resident registration number, service account information, and terminal identification number.

3. The method of claim 1, wherein the acquiring of the counselee information matched with the medical consultation content includes:
acquiring the counselee information through a counselee information input interface provided through a web browser; or
acquiring the counselee information pre-stored through member registration of the medical consultation application.

4. The method of claim 1, wherein the acquiring of the medical consultation content of the counselee through the offline printout includes:
recognizing an identification code included in the offline printout; and
acquiring the medical consultation content matched with the recognized identification code,
wherein the identification code, as a medium for identifying the medical consultation content, is any one of a QR code, a barcode, or a unique identification number.

5. The method of claim 1, wherein the controlling of the display to output the medical consultation content matched with the counselee information further includes:
when the medical consultation application is presently installed in the counselee terminal, outputting the medical consultation content by operating the medical consultation application; or
when the medical consultation application is not installed in the counselee terminal, outputting the medical consultation content in a form of a text or providing an installation process for the medical consultation application.

6. The method of claim 5, further including: providing a member registration interface for the medical consultation application and acquiring the counselee information when installation of the medical consultation application is completed based on the installation process for the medical consultation application.

7. The method of claim 1, wherein the managing of the stored medical consultation content on the basis of the medical consultation application includes providing a medical consultation content management function including classification management, deletion management, and counselee information interlock management for the at least one piece of medical consultation content.

8. The method of claim 7, wherein the managing of the stored medical consultation content on the basis of the medical consultation application further includes providing a member registration interface, wherein the member registration interface is an interface for registering the counselee information for a plurality of counselees by adding at least one counselee other than the counselee to the medical consultation application as members.

9. The method of claim 8, wherein the managing of the acquired medical consultation content on the basis of the medical consultation application further includes providing an owner selection interface, wherein, upon receiving the medical consultation content on the basis of the medical consultation application, the owner selection interface is an interface that matches the received medical consultation content with the counselee information of any one of the plurality of counselees registered in the medical consultation application on the basis of the member registration interface.

10. The method of claim 9, wherein the counselee information interlock management further includes a function of selecting the counselee information matched with each of the medical consultation content existing in the medical consultation application on the basis of the counselee information for the plurality of counselees when the plurality of counselees are registered in the medical consultation application.
